(19) United States
(12) Patent Application Publication
(10) Pub. No.: US 2006/0189495 A1
(43) Pub. Date: Aug. 24, 2006

(54) Compositions with suspended particles

(76) Inventors: Joseph LiBrizzi, Neshanic, NJ (US); Russel M. Walters, Philadelphia, PA (US); Kristen Sweeney, Raritan, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/061,911

(22) Filed: Feb. 18, 2005

(51) Int. Cl. A61K 8/00

(52) U.S. Cl. 510/130

(57) ABSTRACT

Provided are compositions comprising a hydrophobically-modified acrylic polymer, sodium trideceth sulfate, and one or more particles suspended therein having unexpectedly high stability. Also provided are methods of suspending at least one particle comprising combining at least one particle with a hydrophobically-modified acrylic polymer and sodium trideceth sulfate to produce a composition comprising the hydrophobically-modified acrylic polymer and sodium trideceth sulfate in which the at least one particle is suspended.

### FIELD OF INVENTION

The present invention is directed to compositions having particles suspended therein and, more particularly, to compositions in which particles are suspended with unexpectedly high stability and methods of suspending particles in such compositions.

### BACKGROUND

Personal care compositions having beads or other particles suspended therein are desirable conventionally for a variety of uses. Beads or particles tend to impart, or contribute to, a multitude of user benefits associated with personal care compositions including but not limited to: abrasion, visual impact or esthetics, and/or the encapsulation and release of separate phases upon use.

Applicants have nevertheless recognized that the addition of beads or particles to personal care compositions tends to be problematic. For example, one problem recognized by applicants is that particles very frequently tend to be of a different density than the majority phase of the composition to which they are added. This mismatch in the density can lead to separation of the particles from the majority phase and a lack of overall product stability. That is, when added particles are less dense than the composition majority phase, the particles tend to rise to the top of such phase (often referred to in the art as “creaming”). When the added particles have a density greater than the majority phase, the particles tend to fall to the bottom of such phase (often referred to in the art as “settling”). Because applicants believe the driving force of separation is the density mismatch between the particles and the majority phase of composition, as the radius of a particle to be added to the composition increases, the driving force for separation increases, resulting in a particle that is more likely to settle or cream in the composition. Applicants have thus recognized that the apparent relationship of particle size to likelihood of separation makes the stability problem all the more challenging. Since particles are often conventionally supplied with a broad distribution of sizes, the stability of the composition including such particles depends on the stability of the largest particles in the distribution, or the particles that are most difficult to maintain in suspension.

Applicants have recognized that one conventional approach to slowing the separation of particles from compositions is to make the composition more viscous. A variety of polymeric materials, including, for example, hydrophobically-modified polymers (HMPs) have been used conventionally in attempts to thicken and provide suspending ability to various compositions. HMPs tend to form both inter and intra molecular associations with themselves and also with surfactants, which associations create three-dimensional structures that affect rheology, and provide means to suspend particles. Applicants have recognized, however, that merely increasing the viscosity of a composition via the addition of polymers tends only to slow the velocity of the particles and the rate of their separation from the majority phase, rather then prevent or more effectively impede separation. More ideally, applicants have recognized that a better solution would involve modifying the rheology of the formula to suspend the beads such that no separation occurs. Unfortunately, the levels of HMPs required to effectively suspend particles in conventional compositions tends also to impart rheology/aesthetic characteristics to the compositions that are unacceptable from a consumer standpoint.

Accordingly, applicants have identified the need to provide compositions comprising HMPs that not only exhibit the ability to effectively suspend particles therein, but also exhibit desirable rheology/aesthetic characteristics.

### SUMMARY OF INVENTION

According to one aspect, the present invention provides compositions comprising a hydrophobically-modified acrylic polymer, sodium trideceth sulfate, and one or more particles suspended therein.

According to another aspect, the present invention provides methods of suspending a particle comprising combining at least one particle with a hydrophobically-modified acrylic polymer and sodium trideceth sulfate to produce a composition comprising said hydrophobically-modified acrylic polymer and sodium trideceth sulfate in which said at least one particle is suspended.

### DESCRIPTION OF PREFERRED EMBODIMENTS

The present invention overcomes the disadvantages of the prior art by providing compositions comprising HMPs that are capable of forming unexpectedly stable suspensions of particles therein as compared to conventional compositions. In particular, applicants have discovered unexpectedly that certain HMPs can be combined with sodium trideceth sulfate in amounts suitable to produce compositions exhibiting surprisingly high stability for suspending particles therein as compared to conventional compositions and desirable aesthetics for a variety of uses. Accordingly, in certain embodiments, the present invention provides compositions comprising at least one hydrophobically-modified acrylic polymer, sodium trideceth sulfate, and one or more particles suspended therein which compositions are unexpectedly stable and exhibit desirable aesthetics.

With regard to the ability of a composition to suspend particles therein, applicants have recognized that the yield point of a particular composition, as measured via the Oscillatory stress sweep methodology described herein and as commonly understood in the art, is a measure of the ability of a composition to effectively suspend a particle or particles therein. A composition with a yield point tends not to begin to flow until the stress applied to the systems exceeds the yield point and the structure of the system is disturbed. When the stress is below the yield point, the system displays elastic behavior, or ‘solid-like’ behavior. Thus, in general, the higher the yield point of a composition, the greater its ability to suspend particles therein tends to be. Applicants have discovered that the compositions of the present invention tend to have unexpectedly high yield values associated therewith as compared to conventional compositions. In certain preferred embodiments, the present compositions have a yield value of about 4 or greater. In more preferred embodiments, the compositions have a yield value of about 7 or greater, more preferably about 9 or greater, even more preferably about 10 or greater, and even more preferably about 12 or greater.

Applicants have further recognized that certain rheology properties related to aesthetics include the elastic modulus G′, and the viscous modulus, G″, as measured for the purposes of the present invention via the Oscillatory frequency sweep method described further herein, and as understood conventionally in the art. Applicants have discovered that certain preferred, unexpectedly stable, compositions of the present invention also tend to have relatively low G′ and G″ values (desirable aesthetics) associated therewith. In particular, certain preferred compositions exhibit a G′ at 50 rad/s of about 130 or less, more preferably about 120 or less, and even more preferably about 100 or less, and a G″ at 50 rad/s of about 180 or less, more preferably about 160 or less, and even more preferably about 140 or less.

Any of a variety of hydrophobically-modified polymers may be used according to the present invention. As used herein, the term “hydrophobically-modified polymers” refers generally to polymers having one or more hydrophobic moieties attached thereto or incorporated therein. Such polymers may be formed, for example, by polymerizing one or more hydrophobic monomers and, optionally, one or more co-monomers, to form a polymer having hydrophobic moieties incorporated therein, and/or also by reacting polymer materials with compounds comprising hydrophobic moieties to attach such compounds to the polymers. Some hydrophobically-modified polymers and methods of making such polymers are described in U.S. Pat. No. 6,433,061, issued to Marchant et al. and incorporated herein by reference.

Certain preferred hydrophobically-modified polymers for use in the present invention include hydrophobically-modified acrylic polymers. Hydrophobically-modified acrylic polymers suitable for use in the present invention may be in the form of random, block, star, graft copolymers, and the like. In certain embodiments, the hydrophobically-modified acrylic polymers are crosslinked, anionic acrylic copolymers. Such copolymers may be synthesized from at least one acidic monomer and at least one hydrophobic ethylenically unsaturated monomer. Examples of suitable acidic monomers include those ethylenically unsaturated acid monomers that may be neutralized by a base. Examples of suitable hydrophobic ethylenically unsaturated monomers include those that contain a hydrophobic chain having a carbon chain length of at least 3 carbon atoms.

In another embodiment, the hydrophobically-modified, crosslinked, anionic acrylic copolymer includes those compositions derived from at least one unsaturated carboxylic acid monomer; at least one hydrophobic monomer; a hydrophobic chain transfer agent comprising alkyl mercaptans, thioesters, amino acid-mercaptan-containing compounds or peptide fragments, or combinations thereof; a cross-linking agent; and, optionally, a steric stabilizer; wherein the amount of said unsaturated carboxylic acid monomer is from about 60% to about 98% by weight based upon the total weight of said unsaturated monomers and said hydrophobic monomer, as set forth in U.S. Pat. No. 6,433,061, which is incorporated by reference herein. In one embodiment, the polymer is an acrylates copolymer that is commercially available from Noveon, Inc. under the tradename, “Carbopol Aqua SF-1.”

Any suitable amounts of hydrophobically-modified polymers may be used according to the instant invention. In certain preferred embodiments, the compositions of the present invention comprise from about 0.8 to about 30, preferably from about 0.8 to about 15, more preferably from about 1 to about 10, and even more preferably about 1 to about 3 weight percent of hydrophobically-modified polymer. As used herein and throughout, all weight percents refer to weight percent of active material based on the total weight percent of the composition, unless otherwise indicated.

Sodium trideceth sulfate is the sodium salt of sulfated ethoxylated tridecyl alcohol that conforms generally to the following formula, Cl₃H₂₇(OCH₂CH₂)ₙOSO₃Na, where n has a value between 1 and 4. Sodium trideceth sulfate derived from any commercial, synthetic, or other source is suitable for use herein. For example, sodium trideceth sulfate is commercially available from Stepan Company of Northfield, Ill. under the tradename, “Cedapal TD403M.” Applicants have recognized that sodium trideceth sulfate can be used to particular advantage to obtain compositions having significantly stablilized suspensions of particles therein.

Any suitable amount of sodium trideceth sulfate may be used according to the present invention. In certain preferred embodiments, the compositions of the present invention comprise from about 0.1 to about 90, preferably from about 0.1 to about 25, more preferably from about 1 to about 8, and even more preferably about 2 to about 4 weight percent of sodium trideceth sulfate.

Any of a variety of suitable particulate materials may be used as particles for suspension in the present compositions. The type of particles being suspended can include many different morphologies and compositions. The particles can be solid, hollow, or porous. The particles can also encapsulate a phase separate and/or different from the majority phase of the composition. The particles can be comprised of any of a variety of materials including synthetic polymers such as polyethylene, polystyrene, poly gelatins, arabic gums, collagens, polypeptides from vegetable or animal origin, alginates, polyamides, glycosamino glycans, mucopolysaccharides, ethylcellulose, combinations two or more thereof, and the like. Examples of certain commercially available particles include: Jojoba esters particles available from FloraTech (Gilbert, Ariz.) under the trade name Floraspheres, and Florasomes with sizes between 500 to 1500 microns, beads of microcrystalline wax available form FloraTech under the trade name Metabeads, polyethylene particles from Lipo Chemical Inc. (Paterson, N.J.) under the trade name Liposcrubm, walnut shell particles from Lipo Chemical Inc. (Paterson, N.J.) under the trade name Lipo WSF, and the like.

Generally, particles are supplied commercially with a wide distribution of sizes. In certain embodiments, particles suitable for use herein comprise diameters of from about 200 to about 2500 micron. In certain preferred embodiments, the particles have diameters of from about 400 to about 2000 micron, and even more preferably from about 800 to about 1800 micron.

Any suitable amount of particulate matter may be used in the composition of the present invention. Preferably, the present compositions comprise from about 0.1 wt. % to about 10 wt. %, more preferably 0.5 wt. % to 5 wt. %, and most preferably from 0.5 wt. % to 3 wt. % of particulate matter.

The hydrophobically-modified polymers, sodium trideceth sulfate, and particulate matter may be combined according to the present invention via any conventional methods of combining two or more fluids, or two or more fluids with particulate matter, in any order, to suspend the particulate matter therein and achieve a composition of the present invention. For example, a composition of the present invention may be combined by pouring, mixing, adding dropwise, pipetting, pumping, and the like, where appropriate, one or more HMP, sodium trideceth sulfate, and/or one or more particles into or with any other component, in any order, using any conventional equipment such as a mechanically stirred propeller, paddle, glass rod, and the like. According to certain embodiments, the combining step comprises combining a composition comprising sodium trideceth sulfate into or with a composition comprising a hydrophobically-modified polymer, and then adding particulate matter thereto. According to certain other embodiments, the combining step comprises combining a composition comprising a hydrophobically-modified polymer into or with a composition comprising sodium trideceth sulfate and then adding particulate matter thereto. In other preferred embodiments, either one or the other or both of the hydrophobically-modified polymer and sodium trideceth sulfate are added subsequently to a composition comprising the particulate matter

The present compositions produced, as well as any of the compositions comprising hydrophobically-modified polymers, sodium trideceth sulfate, and or particulate matter that are combined in the combining step according to the present methods may further comprise any of a variety of other components nonexclusively including one or more anionic, nonionic, amphoteric, and/or cationic surfactants, pearlescent or opacifying agents, thickening agents, secondary conditioners, humectants, chelating agents, and additives which enhance the appearance, feel and fragrance of the compositions, such as colorants, fragrances, preservatives, pH adjusting agents, and the like.

According to certain embodiments, suitable anionic surfactants include those selected from the following classes of surfactants: alkyl sulfates, alkyl ether sulfates, alkyl monoglyceryl ether sulfates, alkyl sulfonates, alkylaryl sulfonates, alkyl sulfosuccinates, alkyl ether sulfosuccinates, alkyl sulfosuccinamates, alkyl amidosulfosuccinates, alkyl carboxylates, alkyl amidoethercarboxylates, alkyl succinates, fatty acyl sarcosinates, fatty acyl amino acids, fatty acyl taurates, fatty alkyl sulfoacetates, alkyl phosphates, and mixtures of two or more thereof. Examples of certain preferred anionic surfactants include:

alkyl sulfates of the formula
R′—CH₂OSO₃X′;

alkyl ether sulfates of the formula
R′(OCH₂CH₂)ᵥOSO₃X′;

alkyl monoglyceryl ether sulfates of the formula

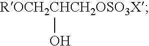

alkyl monoglyceride sulfates of the formula

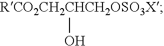

alkyl monoglyceride sulfonates of the formula

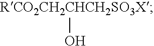

alkyl sulfonates of the formula
R′—SO₃X′;

alkylaryl sulfonates of the formula

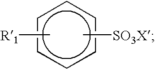

alkyl sulfosuccinates of the formula:

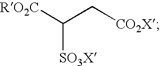

alkyl ether sulfosuccinates of the formula:

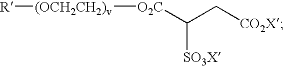

alkyl sulfosuccinamates of the formula:

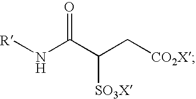

alkyl amidosulfosuccinates of the formula

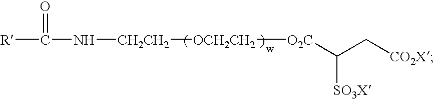

alkyl carboxylates of the formula:
R′—(OCH₂CH₂)_{w}—OCH₂CO₂X′;

alkyl amidoethercarboxylates of the formula:

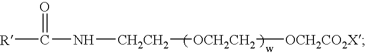

alkyl succinates of the formula:

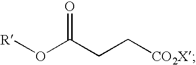

fatty acyl sarcosinates of the formula:

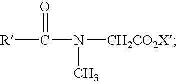

fatty acyl amino acids of the formula:

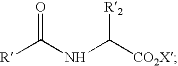

fatty acyl taurates of the formula:

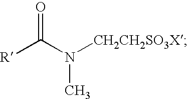

fatty alkyl sulfoacetates of the formula:

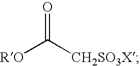

alkyl phosphates of the formula:

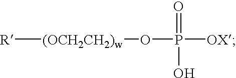

wherein

R′ is an alkyl group having from about 7 to about 22, and preferably from about 7 to about 16 carbon atoms,

R′₁ is an alkyl group having from about 1 to about 18, and preferably from about 8 to about 14 carbon atoms,

R′₂ is a substituent of a natural or synthetic I-amino acid,

X′ is selected from the group consisting of alkali metal ions, alkaline earth metal ions, ammonium ions, and ammonium ions substituted with from about 1 to about 3 substituents, each of the substituents may be the same or different and are selected from the group consisting of alkyl groups having from 1 to 4 carbon atoms and hydroxyalkyl groups having from about 2 to about 4 carbon atoms and

v is an integer from 1 to 6;

w is an integer from 0 to 20;

and mixtures thereof.

Any of a variety of nonionic surfactants are suitable for use in the present invention. Examples of suitable nonionic surfactants include, but are not limited to, fatty alcohol acid or amide ethoxylates, monoglyceride ethoxylates, sorbitan ester ethoxylates alkyl polyglycosides, mixtures thereof, and the like. Certain preferred nonionic surfactants include polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerine, pentaerydiritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester. Examples of such preferred polyoxyethylene derivatives of polyol esters include, but are not limited to PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the tradename, “Atlas G4280.” Polysorbate 20, which is the laurate monoester of a mixture of sorbitol and sorbitol anhydrides condensed with approximately 20 moles of ethylene oxide, is available commercially from ICI Surfactants of Wilmington, Del. under the tradename “Tween 20.”

Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. Preferred alkyl gluocosides comprise from about 1 to about 6 glucose residues per molecule of alkyl glucoside. A preferred glucoside is decyl glucoside, which is the condensation product of decyl alcohol with a glucose polymer and is available commercially from Henkel Corporation of Hoboken, N.J. under the tradename, “Plantaren 2000.”

As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. The amphoteric surfactants are disclosed herein without a counter ion. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions.

Examples of amphoteric surfactants suitable for use in the present invention include, but are not limited to, amphocarboxylates such as alkylamphoacetates (mono or di); alkyl betaines; amidoalkyl betaines; amidoalkyl sultaines; amphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof.

Examples of suitable amphocarboxylate compounds include those of the formula:
A—CONH(CH₂)ₓN⁺R₅R₆R₇

wherein

A is an alkyl or alkenyl group having from about 7 to about 21, e.g. from about 10 to about 16 carbon atoms;

x is an integer of from about 2 to about 6;

R₅ is hydrogen or a carboxyalkyl group containing from about 2 to about 3 carbon atoms;

R₆ is a hydroxyalkyl group containing from about 2 to about 3 carbon atoms or is a group of the formula:
R₈—O—(CH₂)ₙCO₂⁻

wherein

R₈ is an alkylene group having from about 2 to about 3 carbon atoms and n is 1 or 2; and

R₇ is a carboxyalkyl group containing from about 2 to about 3 carbon atoms; Examples of suitable alkyl betaines include those compounds of the formula:
B—N⁺R₉R₁₀(CH₂)ₚCO₂⁻

wherein

B is an alkyl or alkenyl group having from about 8 to about 22, e.g., from about 8 to about 16 carbon atoms;

R₉ and R₁₀ are each independently an alkyl or hydroxyalkyl group having from about 1 to about 4 carbon atoms; and

p is 1 or 2.

A preferred betaine for use in the present invention is lauryl betaine, available commercially from Albright & Wilson, Ltd. of West Midlands, United Kingdom as “Empigen BB/J.”

Examples of suitable amidoalkyl betaines include those compounds of the formula:
D—CO—NH(CH₂)_{q}—N⁺R₁₁R₁₂(CH₂)ₘCO₂⁻

wherein

D is an alkyl or alkenyl group having from about 7 to about 21, e.g. from about 7 to about 15 carbon atoms;

R₁₁ and R₁₂ are each independently an alkyl or

Hydroxyalkyl group having from about 1 to about 4 carbon atoms;

q is an integer from about 2 to about 6; and m is 1 or 2.

One amidoalkyl betaine is cocamidopropyl betaine, available commercially from Goldschmidt Chemical Corporation of Hopewell, Va. under the tradename, “Tegobetaine L7.”

Examples of suitable amidoalkyl sultaines include those compounds of the formula

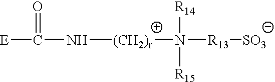

wherein

E is an alkyl or alkenyl group having from about 7 to about 21, e.g. from about 7 to about 15 carbon atoms;

R₁₄ and R₁₅ are each independently an alkyl, or hydroxyalkyl group having from about 1 to about 4 carbon atoms;

r is an integer from about 2 to about 6; and

R₁₃ is an alkylene or hydroxyalkylene group having from

about 2 to about 3 carbon atoms;

In one embodiment, the amidoalkyl sultaine is cocamidopropyl hydroxysultaine, available commercially from Rhone-Poulenc Inc. of Cranbury, N.J. under the tradename, “Mirataine CBS.”

Examples of suitable amphophosphate compounds include those of the formula:

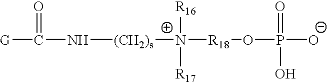

wherein

G is an alkyl or alkenyl group having about 7 to about 21, e.g. from about 7 to about 15 carbon atoms;

s is an integer from about 2 to about 6;

R₁₆ is hydrogen or a carboxyalkyl group containing from about 2 to about 3 carbon atoms;

R₁₇ is a hydroxyalkyl group containing from about 2 to about 3 carbon atoms or a group of the formula:
R₁₉—O—(CH₂)ₜ—CO₂⁻

wherein

R₁₉ is an alkylene or hydroxyalkylene group having from about 2 to about 3 carbon atoms and

t is 1 or 2; and

R₁₈ is an alkylene or hydroxyalkylene group having from about 2 to about 3 carbon atoms.

In one embodiment, the amphophosphate compounds are sodium lauroampho PG-acetate phosphate, available commercially from Mona Industries of Paterson, N.J. under the tradename, “Monateric 1023,” and those disclosed in U.S. Pat. No. 4,380,637, which is incorporated herein by reference.

Examples of suitable phosphobetaines include those compounds of the formula: 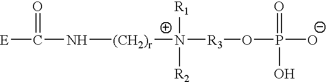
wherein E, r, R₁, R₂ and R₃, are as defined above. In one embodiment, the phosphobetaine compounds are those disclosed in U.S. Pat. Nos. 4,215,064, 4,617,414, and 4,233,192, which are all incorporated herein by reference.

Examples of suitable pyrophosphobetaines include those compounds of the formula:

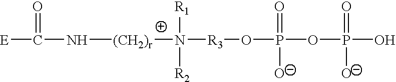

wherein E, r, R₁, R₂ and R₃, are as defined above. In one embodiment, the pyrophosphobetaine compounds are those disclosed in U.S. Pat. Nos. 4,382,036, 4,372,869, and 4,617,414, which are all incorporated herein by reference.

Examples of suitable carboxyalkyl alkylpolyamines include those of the formula:

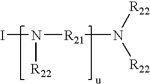

wherein

I is an alkyl or alkenyl group containing from about 8 to about 22, e.g. from about 8 to about 16 carbon atoms;

R₂₂ is a carboxyalkyl group having from about 2 to about 3 carbon atoms;

R₂₁ is an alkylene group having from about 2 to about 3 carbon atoms and

u is an integer from about 1 to about 4.

Classes of cationic surfactants that are suitable for use in this invention include alkyl quaternaries (mono, di, or tri), benzyl quaternaries, ester quaternaries, ethoxylated quaternaries, alkyl amines, and mixtures thereof, wherein the alkyl group has from about 6 carbon atoms to about 30 carbon atoms, with about 8 to about 22 carbon atoms being preferred.

Any of a variety of commercially available pearlescent or opacifying agents which are capable of suspending water insoluble additives such as silicones and/or which tend to indicate to consumers that the resultant product is a conditioning shampoo are suitable for use in this invention. The pearlescent or opacifying agent may be present in an amount, based upon the total weight of the composition, of from about 1 percent to about 10 percent, e.g. from about 1.5 percent to about 7 percent or from about 2 percent to about 5 percent. Examples of suitable pearlescent or opacifying agents include, but are not limited to mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO—(JO)ₐ—H, wherein J is an alkylene group having from about 2 to about 3 carbon atoms; and a is 2 or 3;fatty alcohols containing from about 16 to about 22 carbon atoms; fatty esters of the formula: KCOOCH₂L, wherein K and L independently contain from about 15 to about 21 carbon atoms; inorganic solids insoluble in the shampoo composition, and mixtures thereof

The pearlescent or opacifying agent may be introduced to the mild cleansing composition as a pre-formed, stabilized aqueous dispersion, such as that commercially available from Henkel Corporation of Hoboken, New Jersey under the tradename, “Euperlan PK-3000.” This material is a combination of glycol distearate (the diester of ethylene glycol and stearic acid), Laureth-4 (CH₃(CH₂)₁₀CH₂(OCH₂CH₂)₄OH) and cocamidopropyl betaine and may be in a weight percent ratio of from about 25 to about 30: about 3 to about 15: about 20 to about 25, respectively.

Any of a variety of commercially available thickening agents, which are capable of imparting the appropriate viscosity to the personal cleansing compositions are suitable for use in this invention. If used, the thickener should be present in the shampoo compositions in an amount sufficient to raise the Brookfield viscosity of the composition to a value of between about 500 to about 10,000 centipoise. Examples of suitable thickening agents nonexclusively include: mono or diesters of 1) polyethylene glycol of formula: HO—(CH₂CH₂O)_{z}H, wherein z is an integer from about 3 to about 200; and 2) fatty acids containing from about 16 to about 22 carbon atoms; fatty acid esters of ethoxylated polyols; ethoxylated derivatives of mono and diesters of fatty acids and glycerine; hydroxyalkyl cellulose; alkyl cellulose; hydroxyalkyl alkyl cellulose; and mixtures thereof. Preferred thickeners include polyethylene glycol ester, and more preferably PEG-150 distearate which is available from the Stepan Company of Northfield, Ill. or from Comiel, S.p.A. of Bologna, Italy under the tradename, “PEG 6000 DS”.

Any of a variety of commercially available secondary conditioners, such as volatile silicones, which impart additional attributes, such as gloss to the hair are suitable for use in this invention. In one embodiment, the volatile silicone conditioning agent has an atmospheric pressure boiling point less than about 220° C. The volatile silicone conditioner may be present in an amount of from about 0 percent to about 3 percent, e.g. from about 0.25 percent to about 2.5 percent or from about 0.5 percent to about 1.0 percent, based on the overall weight of the composition. Examples of suitable volatile silicones nonexclusively include polydimethylsiloxane, polydimethylcyclosiloxane, hexamethyldisiloxane, cyclomethicone fluids such as polydimethylcyclosiloxane available commercially from Dow Corning Corporation of Midland, Mich. under the tradename, “DC-345” and mixtures thereof, and preferably include cyclomethicone fluids.

Any of a variety of commercially available humectants, which are capable of providing moisturization and conditioning properties to the personal cleansing composition, are suitable for use in the present invention. The humectant may be present in an amount of from about 0 percent to about 10 percent, e.g. from about 0.5 percent to about 5 percent or from about 0.5 percent to about 3 percent, based on the overall weight of the composition. Examples of suitable humectants nonexclusively include: 1) water soluble liquid polyols selected from the group comprising glycerine, propylene glycol, hexylene glycol, butylene glycol, dipropylene glycol, and mixtures thereof; 2)polyalkylene glycol of the formula: HO—(R″O)_{b}—H, wherein R″ is an alkylene group having from about 2 to about 3 carbon atoms and b is an integer of from about 2 to about 10; 3) polyethylene glycol ether of methyl glucose of formula CH₃—C₆H₁₀O₅—(OCH₂CH₂)_{c}—OH, wherein c is an integer from about 5 to about 25; 4) urea; and 5) mixtures thereof, with glycerine being the preferred humectant.

Examples of suitable chelating agents include those which are capable of protecting and preserving the compositions of this invention. Preferably, the chelating agent is ethylenediamine tetracetic acid (“EDTA”), and more preferably is tetrasodium EDTA, available commercially from Dow Chemical Company of Midland, Mich. under the tradename, “Versene 100XL” and is present in an amount, based upon the total weight of the composition, from about 0 to about 0.5 percent or from about 0.05 percent to about 0.25 percent.

Suitable preservatives include Quaternium-15, available commercially as “Dowicil 200” from the Dow Chemical Corporation of Midland, Mich., and are present in the composition in an amount, based upon the total weight of the composition, from about 0 to about 0.2 percent or from about 0.05 percent to about 0.10 percent.

The methods of the present invention may further comprise any of a variety of steps for mixing or introducing one or more of the optional components described hereinabove with or into a composition comprising a hydrophobically-modified material and/or sodium trideceth sulfate either before, after, or simultaneously with the combining step described above. While in certain embodiments, the order of mixing is not critical, it is preferable, in other embodiments, to pre-blend certain components, such as the fragrance and the nonionic surfactant before adding such components into a composition of the present invention.

The compositions produced via the present invention are preferably used as or in personal care products such as shampoos, washes, baths, gels, lotions, creams, and the like. As discussed above, applicants have discovered unexpectedly that the instant methods allow for the formulation of such personal care products having unexpectedly high stability of suspended particles therein, as well as, preferred aesthetic properties.

### EXAMPLES

### Examples 1-2

### Preparation of Cleansing Compositions for Rheological Measurement

The cleansing compositions of Examples 1 through 2 were prepared according to the materials and amounts listed in Table 1.:

TABLE 1*

| Tradename | INCI Name | 1 | 2 |
|---|---|---|---|
| Plantaren 2000 | Decyl Polyglucose | 2.84 | 2.84 |
| Monateric 1023 | Sodium Lauroampho | 0.90 | 0.90 |
| | PG-Acetate | | |
| | Phosphate | | |
| Glucamate LT | PEG-120 Methyl | 0.27 | 0.27 |
| | Glucose Trioleate | | |
| Glycerin | Glycerin | 5.40 | 5.40 |
| Tegobetaine L7V | Cocamidopropyl | 3.38 | 3.38 |
| | Betaine | | |
| Carbopol AQUA | Carbomer | 1.50 | 1.50 |
| SF-1 | | | |
| Cedepal TD403LD | Sodium Trideceth | — | 3.07 |
| | Sulfate | | |
| Rhodapex ES2K | Sodium Laureth | 3.07 | — |
| | Sulfate | | |
| Jaguar C17 | Guar | 0.45 | 0.45 |
| | Hydroxypropyl- | | |
| | trimonium Chloride | | |
| Dowicil 200 | Quaternium-15 | 0.050 | 0.050 |
| Versene 100XL | Tetrasodium EDTA | 0.263 | 0.263 |
| Sodium Hydroxide | Sodium Hydroxide | As needed | As needed |
| solution (20%) | | | |
| Water | Water | Qs | qs |

*expressed in % w/w active matter

The compositions of Table 1 were prepared as follows:

Water (27.0 parts) was added to a beaker. The Carbopol AQUA SF1 was added to the water while mixing. Once homogenous, the anionic surfactant (Cedepal TD403LD in Example # 1, Rhodapex ES2K in Example #2) was added to the water with mixing. The following ingredients were added thereto independently with mixing until each respective resulting mixture was homogenous: Tegobetaine L7V, Planatem 2000, Monateric 1023, Glycerin and Glucamate LT. In a separate beaker, Jaguar C17 was mixed with 25 parts of water until a homogenous solution was made. The Jaguar C17/Water premix was then added to the main batch and mixed until homogenous. The Dowicil 200, and Versene 100XL were then added to the batch and mixed until homogenous. The pH of the resulting solution was then adjusted with 20% Sodium Hydroxide solution until a final pH of about 6.5 was obtained. The remainder of the water was then added thereto.

Rheoloey Measurement

All rheological measurements were conducted on a TA Instruments AR 2000 Rheometer (New Castle, Del.). Parallel plate geometer with 0° and a diameter of 40 mm was used. The gap between the plates was set to 400 μm. All Theological measurements were performed at 25° C., and a solvent trap was used to minimize evaporation during the experiment.

Oscillatory Stress Sweep

The oscillatory stress was increased from 0.10 Pa to 15920 Pa, while the frequency was held constant at 1.00 Hz. Seven (7) data points where collected over each decade of the oscillatory stress sweep. The yield point is the stress at which the linear-elastic range is exceeded. The yield point was defined in a manner consistent in the art and with, for example, the methodology described in Mezger, The Rheology Handbook, Vincentz Verlag (Hanover, Germany) 2002, pp. 33-36 and 134. That is, from a plot of the natural log (ln) of the stress, ln(stress), and the ln(strain). At low stress, there is a linear relationship between the ln(stress) and the ln(strain). At higher stress, near the yield point, the linear relationship breaks. To determine the yield point a linear relationship is fit to the data at low stress, and a second linear relationship is fit tangent to the region about the yield point. The yield point is defined as the intersection between the two linear equations. The yield points for Examples 1 and 2 are shown in Table 2.

Oscillatory Frequency Sweep

The linear viscoelastic region was determined from the data from the oscillatory stress sweep, and the oscillatory frequency was conducted within the linear viscoelastic region. The oscillatory frequency was increased from 0.001 to 100 Hz, while the oscillatory stress was held constant at 1.00 Pa. Ten (10) Data points were collected over each decade of the oscillatory frequency sweep. G′ & G″ values are shown from selected oscillatory frequencies in Table 2.

TABLE 2

| | Example 1 | Example 2 |
|---|---|---|
| | (SLES) | (TDES) |
| Oscillatory Stress Sweep | | |
| Yield Point (Pa) | 8.4 | 12.2 |
| Oscillatory Frequency Sweep | | |
| G′ (@ 0.01 rad/s) | 0.80 | 1.8 |
| G′ (@ 50 rad/s) | 117 | 118 |
| G″ (@ 50 rad/s) | 154 | 167 |

The yield point of Example 2 (12.2 Pa) is significantly higher (45% higher) than the yield point of Example 1 (8.4 Pa). This larger yield point suggests that Example 2 has a superior ability to suspend particles compared to Example 1.

The elastic modulus, G′, and viscous modulus, G″ were measure over a range of frequencies. Very low frequencies (<0.1 rad/s), or long time scales, reflect how the formula behaves while in the bottle during the shelf life of the product. While higher frequencies (>1 rad/s), or short time scales, reflect how the consumer experiences the product while in use.

As seen in Table 2, at low frequencies (0.01 rad/s) Example 2 has a significantly higher elastic modulus than Example 1, or Example 2 is more solid-like than Example 1 while in the bottle. The higher G′ of Example 2 also suggests that Example 2 has superior ability to suspend particles compared to Example 1.

While at high frequencies (50 rad/s), Example 2 and Example 1 have essentially the same elastic modulus and also nearly the same viscous modulus. At higher frequencies, relevant to in use conditions, Example 1 and Example 2 have essentially the same rheology. The higher frequency rheology is not relevant to the ability to suspend particles. This suggests that in use Example 1 and Example 2 would provide the same experience to consumers.

### Examples 3-4

### Preparation of Cleansing Compositions with Particulate Matter

The cleansing compositions of Examples 3 through 4 were prepared according to the materials and amounts listed in Table 3:

TABLE 3*

| | | Example 3 | Example 4 |
|---|---|---|---|
| Tradename | INCI Name | (SLES) | (TDES) |
| Plantaren 2000 | Decyl Polyglucose | 2.84 | 2.84 |
| Monateric 1023 | Sodium Lauroampho | 0.90 | 0.90 |
| | PG-Acetate | | |
| | Phosphate | | |
| Glucamate LT | PEG-120 Methyl | 0.27 | 0.27 |
| | Glucose Trioleate | | |
| Glycerin | Glycerin | 5.40 | 5.40 |
| Tegobetaine L7V | Cocamidopropyl | 3.38 | 3.38 |
| | Betaine | | |
| Carbopol AQUA | Carbomer | 1.50 | 1.50 |
| SF-1 | | | |
| Cedepal TD403LD | Sodium Trideceth | — | 3.07 |
| | Sulfate | | |
| Rhodapex ES2K | Sodium Laureth | 3.07 | — |
| | Sulfate | | |
| Jaguar C17 | Guar | 0.45 | 0.45 |
| | Hydroxypropyl- | | |
| | trimonium Chloride | | |
| Dowicil 200 | Quaternium-15 | 0.050 | 0.050 |
| Versene 100XL | Tetrasodium EDTA | 0.263 | 0.263 |
| Green STD #369U | Polyethylene | 0.10 | 0.10 |
| Bead | | | |
| Sodium Hydroxide | Sodium Hydroxide | As needed | As needed |
| solution (20%) | | | |
| Water | Water | Qs | qs |

*expressed in % w/w active matter

The compositions of Table 3 were prepared as follows:

Water (27.0 parts) was added to a beaker. The Carbopol AQUA SF1 was added to the water while mixing. Once homogenous, the anionic surfactant (Rhodapex ES2K in Example #3, Cedepal TD403LD in Example #4,) was added to the water with mixing. The following ingredients were added thereto independently with mixing until each respective resulting mixture was homogenous: Tegobetaine L7V, Planatem 2000, Monateric 1023, Glycerin and Glucamate LT. In a separate beaker, Jaguar C17 was mixed with 25 parts of water until a homogenous solution was made. The Jaguar C17/Water premix was then added to the main batch and mixed until homogenous. The Dowicil 200, and Versene 100XL were then added to the batch and mixed until homogenous. The pH of the resulting solution was then adjusted with 20% Sodium Hydroxide solution until a final pH of about 6.5 was obtained. The Green STD #369U beads were then added under gentle mixing until beads were adequately dispersed. The remainder of the water was then added thereto.

Elevated Temperature Stability Assessment:

To assess the bead suspension ability of Examples 3 and 4, two 125 ml glass jars for each Example were filled with product. For each Example, a 125 ml glass jar filled with product was subjected to storage conditions of 50° and 60° C. in an upright position, for 6 hours. Photographs were taken to compare the bead separation in Examples 3 and 4. The photographs of the two jars were divided into six sections of equal height (section 1 on the bottom and section 6 at the top). The beads in each section were counted to quantify the distribution of beads in the jars of each example, shown in Table 4 below:

TABLE 4

| | Example 3 | Example 4 |
|---|---|---|
| | (SLES) | (TDES) |
| Region of jar | (# of beads) | (# of beads) |
| 6 (top) | 78 | 37 |
| 5 | 16 | 12 |
| 4 | 14 | 14 |
| 3 | 4 | 14 |
| 2 | 0 | 16 |
| 1 (bottom) | 1 | 5 |

In Example 3 a majority of the beads have risen to the top of the jar. After aging, the bottom half (regions 1, 2, and 3) of the formula in Example 3 retains only 8.8% of the initial distribution of beads). Example 3 displays nearly fully or complete separation of the beads from the formula. In contrast, Example 4 shows a modest enrichment of the beads at the top of the jar. After aging, the bottom half (regions 1, 2, and 3) of the formula in Example 4 retains 71% of the initial distribution of beads). As mentioned earlier, the particles are supplied with a distribution of sizes. The few particles that separated were most likely the particles on the large end of the distribution. Example 4 displays slight or early separation of the beads from the formula. Example 4 has a superior ability to suspend beads compared to Example 3.

### Examples 5 and 6

### Preparation of Tensiometry Titration Composition

The compositions of Example 5 and 6 were prepared according to the materials and amounts listed in Table 5:

TABLE 5*

| | | 5 | 6 | 7 | 8 |
|---|---|---|---|---|---|
| Tradename | INCI Name | (SLES) | (SLES) | (TDES) | (TDES) |
| Carbopol | Acrylates | 0.0 | 0.167 | 0.0 | 0.167 |
| AQUA SF1 | Copolymer | | | | |
| Sodium | Sodium | As | 0.0 | As | 0.0 |
| Hydroxide | Hydroxide | needed | | needed | |
| solution (20%) | | | | | |
| DI Water | DI Water | Qs | Qs | Qs | Qs |

*expressed in % w/w active

The compositions of Table 5 were prepared as follows:

HPLC grade water (50.0 parts) was added to a beaker. The Carbopol Aqua SF1 in was added to the water with mixing. The pH of the resulting solution was then adjusted with a 20% Sodium Hydroxide solution (as needed) until a final pH of about 7.0 was obtained. The remainder of the water was then added thereto.

Tensiometry Titration Test:

A well-known method to measure the surface tension of surfactant solutions is the Wilhelmy plate method (Holmberg, K.; Jonsson, B.; Kronberg, B.; Lindman, B. Surfactants and Polymers in Aqueous Solution, Wiley & Sons, p. 347). In the method, a plate is submerged into a liquid and the downward force exerted by of the liquid on the plate is measured. The surface tension of the liquid can then be determined based on the force on the plate and the dimensions of the plate. It is also well known that by measuring the surface tension over a range of concentrations the critical micelle concentration (CMC) can then be determined.

There are commercially available Wilhelmy plate method instruments. In the following examples, a Kruss K12 Tensiomter (Kruss USA, Mathews, N.C.) with a platinum Wilhelmy plate used to determine the surface tension of each sample over a range of concentrations. The test can be run either forward or reverse. In either case, a sample vessel contains some initial solution in which the Wilhelmy plate measures the surface tension. Then a second solution is dosed into the sample vessel, stirred, and then probed again with the Wilhelmy plate. The solution initially in the sample vessel before the titration begins, into which the second solution is dosed, will be referred to hereinafter as the initial solution, and the solution that is dosed into the sample vessel during the titration will be referred to hereinafter as the dosing solution, in accordance with the convention used by Kruss USA.

In the forward titration, the concentration of the initial solution is lower than the concentration of the dosing solution. In this example during forward titration tests, the initial solution was HLPC grade water (Fischer Scientific, N.J.), with no surfactant. The dosing solution was a solution of either sodium laureth sulfate (Example 5, 6) or sodium trideceth sulfate (Example 7, 8) and HLPC grade water (Fischer Scientific, N.J.) with a concentration of 5750 mg/L. A large stock solution, 4L, of each dosing surfactant solution was prepared before hand; sodium laureth sulfate (Stepan Company, Northfield, Ill.) or sodium trideceth sulfate (Stepan Company, Northfield, Ill.) was added to HLPC grade water (Fischer Scientific, N.J.) to a concentration of 5750 mg/L.

At the beginning of the forward titration, 50 ml of initial solution was added to the sample vessel. The surface tension of this initial solution was measured, and then a volume of the dosing solution was added to the sample vessel. The solution was stirred for at least 5 minutes, before the next surface tension measures was taken. All titrations were run from 0 mg/L to at least 3500 mg/L of sodium laureth sulfate or sodium trideceth sulfate, which is well beyond the CMC of all samples. A test run according to this procedure is here after referred to as a Forward Titration Tensiometry Test.

Critical Micelle Concentration Values: The compositions prepared in accordance with Examples 5, 6 7 and 8 were tested for Critical Micelle Concentration (CMC) values using the forward titration tensiometry experiment. The initial solution was 50 ml. The dosing solution was 5750 mg/L of sodium laureth sulfate or sodium trideceth sulfate and HPLC grade water. 42 dose were performed, which increased the sodium trideceth concentration from 0 mg/L in the initial solution up to 3771 mg/L at the final measurement.

The results of this test are listed below in Table 6:

TABLE 6
Critical Micelle Concentration Comparison

| Example Number | CMC value (mg/L) | Delta CMC (mg/L) |
|---|---|---|
| Example 5 (SLES) | 41 | n.a. |
| Example 6 (SLES) | 150 | 109 |
| Example 7 (TDES) | 125 | n.a. |
| Example 8 (TDES) | 400 | 275 |

The CMC is the surfactant concentration (sodium laureth sulfate in Examples 5 and 6 and sodium trideceth sulfate in Examples 7 and 8) at which free micelles begin to form. At surfactant concentration below the CMC, no surfactant exist as free micelles, while at concentrations above the CMC free micelles are present in solution. In Example 8, free micelles begin to form at 400 mg/L of trideceth sulfate.

We believe that the shift in the CMC to higher concentration with the addition of certain materials (i.e., HMP in Example 6 and 8) occurs because surfactant associates with said material, thereby reducing the free monomer concentration. The free monomer concentration is reduced proportional to the amount of surfactant associated with the material. The magnitude of the Delta CMC suggests the amount of surfactant that the material is capable of associating with, or the efficiency of the material in associating surfactant.

In Example 8, the concentration of Carbopol Aqua SF-I was 500 mg/L, and the CMC was 400 mg/L of sodium trideceth sulfate, while the CMC of sodium trideceth sulfate without SF-1 was 125 mg/L. Therefore, the material of Example 8 associated with 275 mg of sodium trideceth sulfate per every 500 mg of material, or 0.55 g of sodium trideceth sulfate per 1.0 g of Aqua SF-1. While in Example 6 the same concentration of Carbopol Aqua SF-1 produced a CMC shift of only 109 mg/L of sodium laureth sulfate. Therefore, the material of Example 6 associated with 109 mg of sodium laureth sulfate per every 500 mg of material, or 0.22 g of sodium trideceth sulfate per 1.0 g of Aqua SF-1. The efficiency of a material to associate surfactant is the Delta CMC per mass of the material. A material with a higher efficiency will associate more surfactant and will produce a larger Delta CMC. We believe that this ability to associate with more surfactant is responsible to the differences observed in the rheological behavior between the sodium laureth sulfate and sodium trideceth sulfate surfactant systems, and there differences observed in the ability to suspend particles.

What is claimed is:

1. A composition comprising a hydrophobically-modified acrylic polymer, sodium trideceth sulfate, and one or more particles suspended therein.

2. The composition of claim 1 wherein said composition has a yield value of about 7 or greater.

3. The composition of claim 1 wherein said composition has a yield value of about 9 or greater.

4. The composition of claim 1 wherein said composition has a yield value of about 12 or greater.

5. The composition of claim 1 having a G′ at 50 rad/s of about 130 or less.

6. The composition of claim 5 having a G′ at 50 rad/s of about 120 or less.

7. The composition of claim 1 having a G″ at 50 rad/s of about 180 or less.

8. The composition of claim 7 having a G″ at 50 rad/s of about 160 or less.

9. The composition of claim 1 wherein said hydrophobically-modified acrylic polymer is derived from at least one unsaturated carboxylic acid monomer; at least one hydrophobic monomer; a hydrophobic chain transfer agent comprising one or more alkyl mercaptans, thioesters, amino acid-mercaptan-containing compounds, peptide fragments, or combinations thereof; a cross-linking agent; and, optionally, a steric stabilizer; wherein the amount of said unsaturated carboxylic acid monomer is from about 60% to about 98% by weight based upon the total weight of said unsaturated monomers and said hydrophobic monomer.

10. The composition of claim 1 comprising from about 0.8 to about 30 weight percent of hydrophobically-modified acrylic polymer.

11. The composition of claim 1 comprising from about 0.8 to about 15 weight percent of hydrophobically-modified acrylic polymer.

12. The composition of claim 1 comprising from about 1 to about 10 weight percent of hydrophobically-modified acrylic polymer.

13. The composition of claim 1 comprising from about 0.1 to about 90 weight percent of sodium trideceth sulfate.

14. The composition of claim 1 comprising from about 0.1 to about 25 weight percent of sodium trideceth sulfate.

15. The composition of claim 1 comprising from about 1 to about 8 weight percent of sodium trideceth sulfate.

16. The composition of claim 1 wherein said one or more particles comprises particles having a diameter of from about 200 to about 2500 micron.

17. The composition of claim 1 wherein said one or more particles comprises particles having a diameter of from about 400 to about 2000 micron.

18. The composition of claim 1 wherein said one or more particles comprises particles having a diameter of from about 800 to about 1800 micron.

19. The composition of claim 1 further comprising one or more materials selected from the group consisting of nonionic, amphoteric, and cationic surfactants, pearlescent agents, opacifying agents, thickening agents, secondary conditioners, humectants, chelating agents, colorants, fragrances, preservatives, and pH adjusting agents.

20. A composition of claim 1 comprising from about 1 to about 3 weight percent of hydrophobically modified acrylic polymer, from about 2 to about 4 weight percent sodium trideceth sulfate, and from about 0.5 to about 5 weight percent of particles.

21. A personal care product comprising a composition of claim 1.

22. A method of suspending a particle comprising combining at least one particle with a hydrophobically-modified acrylic polymer and sodium trideceth sulfate to produce a composition comprising said hydrophobically-modified acrylic polymer and sodium trideceth sulfate in which said at least one particle is suspended.

23. The method of claim 21 wherein said composition has a yield value of about 7 or greater.

24. The method of claim 21 wherein said composition has a yield value of about 9 or greater.

25. The method of claim 21 wherein said hydrophobically-modified acrylic polymer is derived from at least one unsaturated carboxylic acid monomer; at least one hydrophobic monomer; a hydrophobic chain transfer agent comprising one or more alkyl mercaptans, thioesters, amino acid-mercaptan-containing compounds, peptide fragments, or combinations thereof; a cross-linking agent; and, optionally, a steric stabilizer; wherein the amount of said unsaturated carboxylic acid monomer is from about 60% to about 98% by weight based upon the total weight of said unsaturated monomers and said hydrophobic monomer.

26. The method of claim 21 wherein said composition comprises from about 0.8 to about 15 weight percent of hydrophobically-modified acrylic polymer.

27. The method of claim 21 wherein said composition comprises from about 1 to about 10 weight percent of hydrophobically-modified acrylic polymer.

28. The method of claim 21 wherein said composition comprises from about 0. 1 to about 25 weight percent of sodium trideceth sulfate.

29. The method of claim 21 wherein said composition comprises from about 1 to about 8 weight percent of sodium trideceth sulfate.

30. The method of claim 21 wherein said at least one particle comprises particle having a diameter of from about 400 to about 2000 micron.

31. The method of claim 21 wherein said at least one particle comprises particle having a diameter of from about 800 to about 1800 micron.
